(19) United States
(12) Patent Application Publication
(10) Pub. No.: US 2008/0087285 A1
(43) Pub. Date: Apr. 17, 2008

(54) Adjustable multi-functional carrying strap for an ambu bag

(76) Inventor: Tzong-Fuh Kuo, Hsin Juang City (TW)

Correspondence Address: PRO-TECHTOR INTERNATIONAL SERVICES, 20775 NORADA CT., SARATOGA, CA, 95070, US

(21) Appl. No.: 11/581,104

(22) Filed: Oct. 13, 2006

(51) Int. Cl. A62B 7/00; A61M 16/00

(52) U.S. Cl. 128/205.13; 128/202.13

(57) ABSTRACT

An adjustable multi-functional carrying strap for an ambu bag is fixed on the ambu bag, and can be adjusted to be tighter or looser. The carrying strap allows a single palm to be fastened on the ambu bag, such that the ambu bag can be operated with one hand, and a mask can be operated by the other hand to press on a muzzle portion of a patient, so as to effectively achieve a purpose of rescuing. In addition, the carrying strap can also allow the ambu bag to be carried by one hand or to be hung on a wall for backup.

### BACKGROUND OF THE INVENTION

a) Field of the Invention

The present invention relates to an adjustable multi-functional carrying strap for an ambu bag, and more particularly to a multi-functional carrying strap by which an ambu bag can be operated with one hand, and can be carried or hung on a wall conveniently.

b) Description of the Prior Art

As an end of a conventional ambu bag is connected to an oxygen tube, and the other end is connected to a mask, when a patient does not breathe comfortably, a user will use one hand to press a mask on a muzzle portion of the patient, and use the other hand to squeeze the ambu bag to supply oxygen to the patient. However, as the ambu bag is too big, it cannot be held by one hand at all, such that it will be very difficult for one person to carry out a rescuing work, thereby affecting a timing of the rescue. Accordingly, two persons must carry out the rescuing work at a same time, which will waste manpower.

Referring to FIG. 1, an end of a conventional ambu bag 100 is connected to an oxygen tube 200 or an oxygen bag 300, and the other end is connected to a mask 400, whereas the ambu bag 100 is provided with a carrying strap 101. As the carrying strap 101 is fixed and cannot be adjusted, it is only provided with a function of carrying with hands and cannot achieve a purpose of holding the ambu bag 100 with one hand. Accordingly, an improvement is required.

### SUMMARY OF THE INVENTION

The primary object of present invention is to provide a carrying strap which is installed on an ambu bag and can be adjusted for its tightness, such that a user can use one hand to operate the ambu bag to supply oxygen to a patient, while using the other hand to press a mask, thereby allowing one person to effectively carry out a rescuing work.

Another object of the present invention is to provide a carrying strap which can facilitate a carrying of the ambu bag with hands and also allow the ambu bag to be hung on a wall for backup.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a conventional elevational view of a prior art.

FIG. 2 shows a perspective view of the present invention.

FIG. 3 shows a partial structional view of the present invention.

FIG. 4 shows a partial exploded elevational view of the present invention.

FIG. 5 shows another schematic view illustrating actions of the present invention.

FIG. 6 shows a perspective view of an ambu bag being hung on a wall by the present invention.

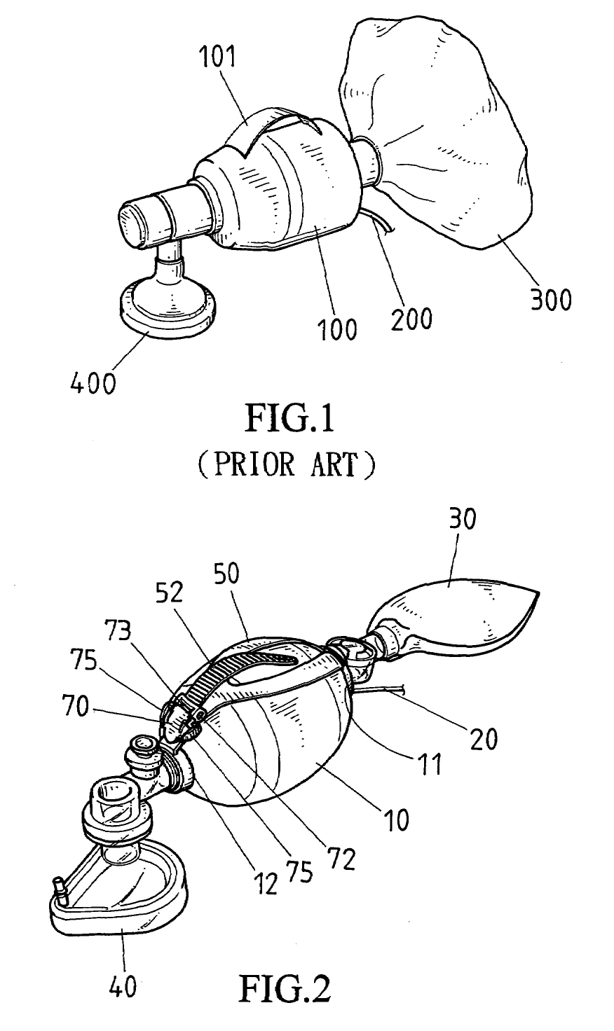

### DETAILED DESCRIPTION OF THE PREFERRED EMBODIMENTS

Referring to FIGS. 2 to 4, an air-inlet valve connector 11 at an end of an ambu bag 10 is connected to an oxygen tube 20 and an oxygen bag 30, and a tube connector 12 at the other end is connected to a mask 40, wherein an outer ring of the air-inlet valve connector 11 is in a gear shape.

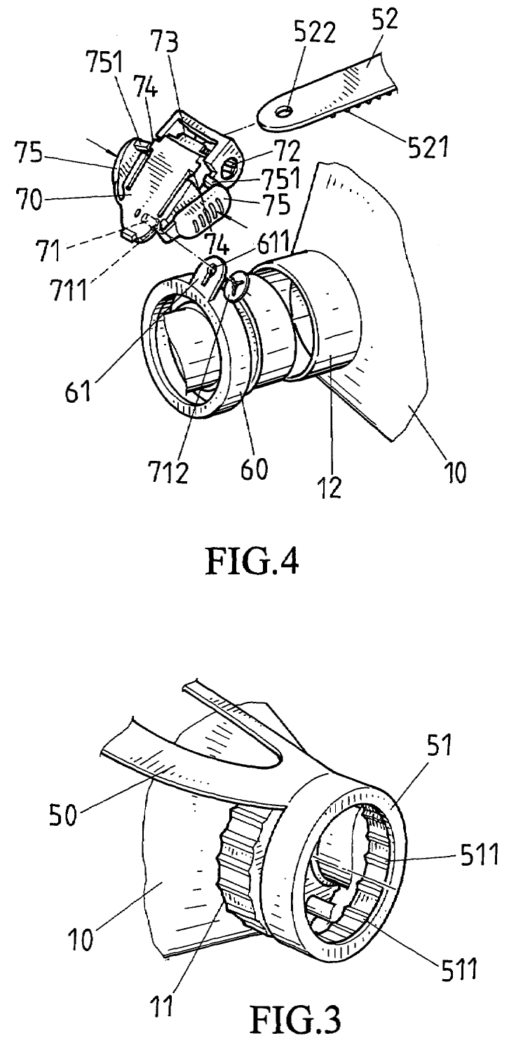

Referring to FIG. 3, the ambu bag 10 is installed with an adjustable carrying strap 50, with an end of which being provided with a circular loop 51. An interior of the loop 51 is provided with an inner ring which is surrounded with a plurality of inner teeth 511. When the loop 51 is sheathed on the air-inlet valve connector 11 of ambu bag 10, the inner teeth 511 of loop 51 are gnawed with the outer ring of air-inlet valve connector 11.

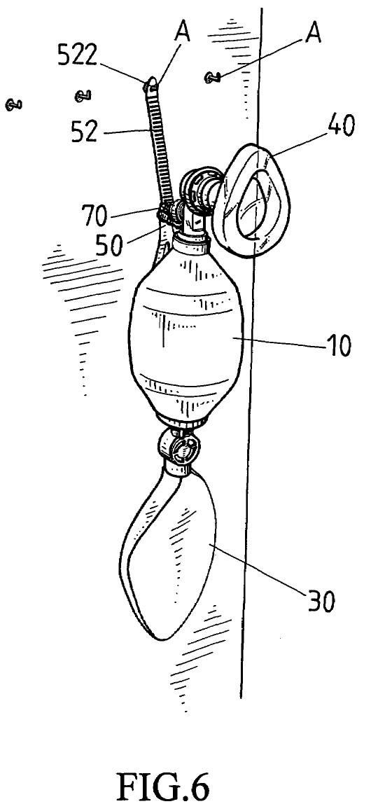

Referring to FIG. 4, a tightening strap 51 at the other end of carrying strap 50 is provided with an entire row of inverted teeth 521 in a slant direction, and a tail end of the tightening strap 52 is provided with a hook hole 522, enabling the ambu bag 10 to be hung on a hook A of wall (as shown in FIG. 6). The tube connector 12 of ambu bag 10 is sheathed with a locking loop 60 to be assembled with the carrying strap 50, and the locking loop 60 which is made by a soft material is provided with a slanted projection block 61. The projection block 61 is provided with a positioning hole 611 in a shape of a trumpet, and a bottom of an interior side of a hard locking member 70 made by a plastic material is provided with a locking button 71 to be locked into the positioning hole 611 of projection block 61. A cone-shape head 711 at a front end of the locking button 71 is locked on a locking device 712 after penetrating the positioning hole 611, so as to rigidly fix the locking member 70 on the projection block 61. An interior side at a top end of the locking member 70 is pivoted with a shaft 72, with a notch between the shaft 72 and the locking member 70 being penetrated by the tightening strap 52. A front end of the locking member 70 is provided with a locking block 73, with locking positions (not shown in the drawing) at a bottom of which being locked into the inverted teeth 521 of tightening strap 52. Therefore, the tightening strap 52 can be only pulled outward to tighten the strap, and cannot be pulled inward to release the strap. Two sides of the locking member 70 that are close to the locking block 73 are provided with a slant surface 74 respectively, and two sides at a bottom of the locking member 70 are extended upward with an elastic push button 75, respectively. An interior side of the push button 75 is transversally provided with a push block 751, such that when a user uses fingers to press the two push buttons 75 inward, the push blocks 751 can extend to the slant surfaces 74 and push up the locking block 73. Therefore, the locking block 73 can be escaped from the locking of inverted teeth 521 of tightening strap 52, and the carrying strap 50 can be freely adjusted to be looser or tighter.

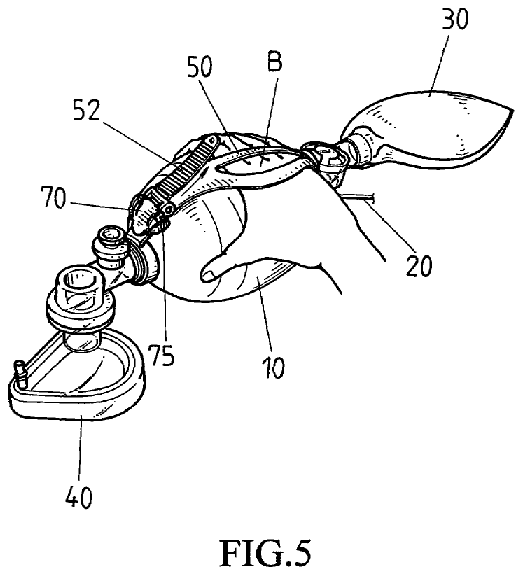

Referring to FIG. 5, a palm B of one hand of a user is extended into the loop of carrying strap 50, and the hand is used to pull the tightening strap 52 to adjust the tightness of strap, such that the palm B can be fastened on the ambu bag 10, thereby enabling the user to use one hand to operate the ambu bag 10.

In summary, the present invention relates to a carrying strap which allows one hand of a user to operate an ambu bag, while enabling the other hand of user to press a mask on a muzzle portion of a patient, thereby enabling one person to effectively carry out the rescuing work. In addition, the user can also hold the carrying strap with one hand to carry the ambu bag, and the ambu bag can be hung on a wall hook for backup when it is not used.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. An adjustable multi-functional carrying strap for an ambu bag, with an air-inlet valve connector at one end of the ambu bag being connected with an oxygen tube and an oxygen bag, and a tube connector at the other end being connected with a mask, wherein an outer ring of the air-inlet valve connector is in a gear shape; the ambu bag being installed with an adjustable carrying strap which comprises a loop, an interior of which is provided with an inner ring which is surrounded with a plurality of inner teeth, and which is sheathed on the air-inlet valve connector of ambu bag, with the inner teeth being gnawed with the outer ring of air-inlet valve connector; a tightening strap, on which is provided with a plurality of inverted teeth in a slant direction; a locking loop, which is sheathed on the tube connector of ambu bag, and which is provided with a slanted projection block having a positioning hole; and a locking member, a bottom of an interior side of which is provided with a locking button being locked into the positioning hole of locking block, with a cone-shape head at a front end of the locking button being locked on a locking device after penetrating the positioning hole, and with an inner side at a top end of the locking member being pivoted with a shaft, whereas a notch between the shaft and the locking member being penetrated by the tightening strap, and a front edge of the locking member being provided with a locking block, with locking positions at a bottom of the locking block being locked into the inverted teeth of tightening strap, with two sides of the locking block being provided with a slant surface respectively, and with two sides at a bottom of the locking block being extended respectively upward with an elastic push button being transversally provided with a push block in an interior; when a user pressing the two push buttons inward, the push blocks extending to the slant surfaces and pushing up the locking block, such that the locking block is escaped from the locking of inverted teeth of tightening strap, thereby freely tightening or loosening the carrying strap.

2. The adjustable multi-functional carrying strap for an ambu bag as claimed in claim 1, wherein a tail end of the tightening strap of carrying strap is provided with a hook hole, such that the ambu bag is hung on a hook of a wall.
